(19) United States
(12) Patent Application Publication
(10) Pub. No.: US 2024/0339021 A1
(43) Pub. Date: Oct. 10, 2024

(54) SCALE TRIGGERING OF EMISSIONS DETECTION, LOCALIZATION, QUANTIFICATION AND REPAIR

(71) Applicant: SeekOps Inc., Austin, TX (US)

(72) Inventors: Iain Cooper, Canyon Lake, TX (US); Michael Price McGuire, Austin, TX (US); Garrett Niall John, Buda, TX (US); Santiago Vigil, Austin, TX (US); Brendan James Smith, Lakeway, TX (US)

(21) Appl. No.: 18/293,196

(22) PCT Filed: Jul. 29, 2022
(86) PCT No.: PCT/US22/38951

§ 371 Date: Jan. 29, 2024

Related U.S. Application Data
(60) Provisional application No. 63/227,800, filed on Jul. 30, 2021.

(51) Int. Cl. G08B 21/12; G08B 27/00; G08B 29/18

(52) U.S. Cl. CPC G08B 21/12; G08B 27/005; G08B 29/185

(57) ABSTRACT

Systems, devices, and methods for an emissions characterization system comprising: one or more trace gas emission monitoring devices; where the one or more trace gas emission detection devices are configured to be connected via a communication network; and where the one or more emission detection devices are further configured to perform one or more of: alert, verify, and quantify anomalous trace gas emissions.

### CROSS-REFERENCE TO RELATED APPLICATION

This application is a 35 U.S.C § 371 National Stage Entry of International Application No. PCT/US22/38951, filed Jul. 29, 2022, which claims the priority benefit of U.S. Provisional Patent Application Ser. No. 63/227,800 filed Jul. 30, 2021, all of which are hereby incorporated herein by reference in their entirety for all purposes.

### FIELD OF ENDEAVOR

Embodiments relate generally to trace gas emissions, and more particularly to detecting and quantifying trace gas emissions

### BACKGROUND

Oil and Gas, renewable natural gas, and waste management industries have all made a concerted effort to limit and offset anthropogenic sources of trace gases such as methane emissions. Additionally, oil and gas operators specifically have pledged to have net zero carbon operations targets.

### SUMMARY

An embodiment disclosed herein comprises a meshed emissions characterization system network comprising autonomous trace gas measurement devices, such as satellites, unmanned aerial vehicles, ground/surface robotics, and fixed monitors. Said devices work in concert to alert, verify, and quantify anomalous trace gas emissions activities through a combined wired and wireless network.

A method embodiment may include: detecting one or more system anomalies; deploying one or more Unmanned Aerial Vehicles (UAVs), wherein each UAV comprises one or more gas sensors configured to detect a presence of one or more gas emissions; alarming or clearing an emissions alarm if emissions are/are not detected by the one or more trace gas sensors of the one or more UAVs; communicating with an industrial network (i.e. Supervisory Control And Data Acquisition (SCADA) system) if emissions are detected by the one or more trace gas sensors of the one or more UAVs; and repairing one or more equipment types generating the detected emissions by at least one of: initiating a repair protocol, which may entail the shutdown of components/equipment, equipment groups, or an entire facility.

Another method embodiment may include: detecting emissions by at least one of: one or more satellite emissions surveys, one or more gas/plume imaging manned or unmanned aircraft emissions surveys, and one or more fixed gas sensor(s) continually monitoring for leaks; triggering a high-resolution sensor deployment; filing the detected emissions as a false positive if the triggered high-resolution sensor deployment does not detect any further emissions; performing localization and quantification of the emissions if the triggered high-resolution sensor deployment detects further emissions; and reconfiguring at least one of: a mesh network sensitivity and a mesh network density if the triggered high-resolution sensor deployment detects further emissions.

Another method embodiment may include: receiving emissions data from at least one of: one or more satellite emissions surveys, one or more surveys using an emission sensor deployed in a manned aircraft or helicopter, and/or a distributed fixed sensor mesh; generating a database comprising at least one of: geographical regions that have higher than average concentrations/leaks/emissions, equipment groups that have higher than average concentrations/leaks/emissions, equipment components that have higher than average concentrations/leaks/emissions, and/or equipment OEMs that have higher than average concentrations/leaks/emissions, determining predictive maintenance from the generated database to minimize future emissions; and determining at least one of: equipment groups and areas that will be more likely to emit trace gasses.

An emissions characterization system may include: one or more trace gas emission monitoring devices where; where the one or more trace gas emission detection devices may be configured to be connected via a communication network; and where the one or more emission detection devices may be further configured to perform one or more of: alert, verify, and quantify anomalous trace gas emissions.

In additional system embodiments, the communication network comprises a mesh network. In additional system embodiments, the mesh network comprises a wired and wireless network. In additional system embodiments, the one or more trace gas emission monitoring devices comprise one or more satellite monitoring systems where the one or more satellite monitoring systems may be configured to generate data from one or more satellites.

In additional system embodiments, the one or more trace gas emission monitoring devices comprise one or more aerial vehicle measurement systems where the one or more aerial vehicle measurement systems comprise one or more trace gas sensors configured to generate data on trace gas concentrations. In additional system embodiments, the one or more aerial vehicle measurement systems comprise a manned aerial vehicle. In additional system embodiments, the one or more aerial vehicle measurement systems comprise an unmanned aerial vehicle (UAV).

In additional system embodiments, the one or more trace gas emission monitoring devices comprise one or more fixed monitor measurement systems where the one or more fixed monitor measurement systems comprise one or more trace gas sensors configured to generate data on trace gas concentrations, and where the one or more trace gas sensors may be fixed in a location. In additional system embodiments, the one or more trace gas emission monitoring devices comprise one or more robotics measurement systems where the one or more robotics measurement systems comprise one or more trace gas sensors configured to generate data on trace gas concentrations. In additional system embodiments, the one or more robotics measurement systems comprise one or more ground robotics. In additional system embodiments, the one or more robotics measurement systems comprise one or more surface robotics.

In additional system embodiments, the one or more trace gas emission monitoring devices comprise one or more autonomous gas measurement systems where the one or more autonomous gas measurement systems comprise one or more trace gas sensors configured to generate data on trace gas concentrations.

Additional system embodiments may further include: an emissions characterization module configured to receive data from the communication network comprising the one or more trace gas emission monitoring devices where. In additional system embodiments, the emissions characterization module may be configured to alert, verify, and quantify anomalous trace gas emissions activities. In additional system embodiments, the emissions characterization module may be configured to determine if the anomalous trace gas emissions activities may be at least one of: a false positive and a result of a trace gas leak. In additional system embodiments, the emissions characterization module may be configured to provide a feedback loop to the communication network comprising the one or more trace gas emission monitoring devices where based on the determination to increase an accuracy of the communication network and reduce a number of false positives. In additional system embodiments, the emissions characterization module may be configured to at least one of: shut off a gas flow and initiate a repair protocol if the determined anomalous trace gas emissions activities may be the result of a trace gas leak. In additional system embodiments, the emissions characterization module may be configured to activate an emissions alarm if the determined anomalous trace gas emissions activities may be the result of a trace gas leak.

A method embodiment may include: detecting emissions of one or more trace gasses by at least one of: one or more satellite emissions surveys one or more distributed sensors in a mesh continually monitoring for leaks and one or more manned aircraft emission surveys; triggering a high-resolution sensor deployment in response to detected emissions of the one or more trace gasses; categorizing the detected emissions of the one or more trace gasses as a false positive if the triggered high-resolution sensor deployment does not detect any further emissions; performing localization and quantification of the emissions if the triggered high-resolution sensor deployment detects further emissions; and reconfiguring at least one of: a mesh network sensitivity and a mesh network density if the triggered high-resolution sensor deployment detects further emissions.

In additional method embodiments, the triggered high-resolution sensor deployment further comprises a high-resolution survey from an unmanned aerial vehicle (UAV) comprising one or more trace gas sensors.

Another method embodiment may include: receiving emissions data from at least one of: one or more satellite emissions surveys a distributed sensor mesh and one or more manned aircraft emission surveys; and generating a database comprising at least one of: geographical regions that have higher than average trace gas emissions, equipment groups that have higher than average trace gas emissions, equipment components that have higher than average trace gas emissions, and equipment manufacturers that have higher than average trace gas emissions.

Additional method embodiments may include: determining predictive maintenance from the generated database to reduce future trace gas emissions. Additional method embodiments may include: determining at least one of: equipment groups and areas that will be more likely to emit trace gasses based on the generated database.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1 depicts an industrial network system for production management, according to one embodiment;

FIG. 2 depicts a multi-well production management system, according to one embodiment;

FIG. 3 depicts a digital workflow, according to one embodiment;

FIG. 4 depicts a workflow for automated emission detection and repair with an integrated industrial network system, according to one embodiment;

FIG. 5 depicts differing scales for emissions monitoring, according to one embodiment;

FIG. 6A depicts a workflow for using differing sensor scales to refine uncertainty in emissions monitoring and reduction, according to one embodiment;

FIG. 6B depicts a workflow for using a closed loop feedback to probability of detection in emissions monitoring and reduction, according to one embodiment;

FIGS. 7A-7C depict various communication systems for communications between one or more sensors and a cloud server;

FIG. 8 depicts a hierarchical meshed emissions characterization system 800, according to one embodiment;

FIG. 9 illustrates an example top-level functional block diagram of a computing device embodiment;

FIG. 10 shows a high-level block diagram and process of a computing system for implementing an embodiment of the system and process;

FIG. 11 shows a block diagram and process of an exemplary system in which an embodiment may be implemented;

FIG. 12 depicts a cloud computing environment for implementing an embodiment of the system and process disclosed herein; and

FIG. 13 depicts a system for detecting trace gasses, according to one embodiment.

### DETAILED DESCRIPTION

The following description is made for the purpose of illustrating the general principles of the embodiments discloses herein and is not meant to limit the concepts disclosed herein. Further, particular features described herein can be used in combination with other described features in each of the various possible combinations and permutations. Unless otherwise specifically defined herein, all terms are to be given their broadest possible interpretation including meanings implied from the description as well as meanings understood by those skilled in the art and/or as defined in dictionaries, treatises, etc.

An embodiment of the disclosed invention herein is directed to detecting emissions of trace gases such as methane, carbon dioxide, and other volatile organic compounds, and also accurately locate the trace gas sources and quantify the amount of the emissions (e.g., leaks). The emissions can be highly variable and depend on operational, equipment and environmental factors, and thus require precision, and validated measurement techniques disclosed according to one or more embodiments herein. Embodiments herein further address evolving regulations around the mitigation of trace gas emissions including detection and quantification.

Embodiments herein ameliorate existing methods for monitoring emissions from oil and gas production operations that have typically employed optical gas imaging (OGI) cameras or handheld ‘sniffer’ systems on an intermittent basis to document leaks for repair. Such surveys can have a subjective bias, are conditional on the skill of the camera operator and give scant indication of a quantifiable leak rate.

An embodiment disclosed herein comprises a trace gas emissions characterization system comprising a network of autonomous trace gas measurement devices, wherein said devices work in concert to alert, verify, and quantify anomalous trace gas emissions activities through communication via wired and/or wireless networks. Another embodiment disclosed herein comprises a hierarchical meshed emissions characterization system comprising autonomous trace gas measurement devices, such as satellites, unmanned aerial vehicles, ground/surface robotics, and fixed monitors. Said devices work in concert to alert, verify, and quantify anomalous trace gas emissions activities through a combined wired and wireless network.

According to one embodiment disclosed herein, satellite based emissions detection and monitoring spectroscopy systems, manned aircraft and helicopter platforms with LiDAR and laser-reflectivity based systems, drone based-OGI and spectroscopy platforms, and fixed-in-place continuous measurements may be used to enhance both the spatial and temporal coverage of industrial operations. These systems may all have differing sensitivities, accuracies, ease of deployment and cost, and all have differing capabilities, and associated limitations. For example, satellites may have low trace gas emission (e.g., trace gas leak) rate resolution, but very large areal coverage. With a constellation of satellites one can get a revisit of a particular location every few days. Manned aircraft and helicopters or larger Beyond Visual Line of Sight (BVLOS) unmanned vehicles may have improved spatial and temporal resolution and may also tend to have improved minimum gas leak detection thresholds relative to satellite systems. Drone-based systems may have further improved minimum detection levels, and have the advantage of being able to fly more proximate to any potentially leaking equipment, and also access potentially difficult to reach or see compared to a ground-based Optical Gas Imaging (OGI) operator, allowing for the full three-dimensionality of any gas plume, Continuously measuring fixed sensors can have, particularly if there is a low density, or if they are inappropriately spaced in three dimensions, issues with localizing and quantifying leaks, but can indicate that a leak has occurred.

These differing spatial and temporal scales need to be addressed with varying instrument sensitivity and accuracy. One or more of these measurement technologies may be utilized to optimize and better refine spatial and temporal coverage of emissions, enabling detection, localization and quantification to levels acceptable for regulatory reporting, and to facilitate the prompt repairs of the leak.

Embodiment of the systems and methods disclosed herein may include a hierarchical meshed emissions characterization system including autonomous gas measurement systems, such as satellites, unmanned aerial vehicles, ground robotics, surface robotics, and fixed monitors. These devices work in concert to alert, verify, and quantify anomalous emissions activities through a combined wired and wireless network.

An embodiment of the systems and methods described herein are in the field of detection of gas emissions from industrial and waste management operations and relate to methods to detect, localize and quantify those emissions. The systems and methods disclosed herein utilize industrial networks (e.g., Supervisory Control and Data Acquisition Systems (SCADA)) and emissions data for automated emissions verification, localization and quantification with subsequent trigger and validation of repairs. A SCADA system is a type of industrial network. In some embodiments, the industrial network may be any network. In some embodiments, the industrial network may include a sensor that communicates to an external network, e.g. a cell tower, to the Internet and then back into their intranet for processing and control.

Techniques for measurements of gas emissions, particularly those that are considered greenhouse gases, contributing to global warming/climate change, such as methane and carbon dioxide encompass a number of different types and form factors or modes of operation. In one example, key measurements are of gas concentration in air. These measurements may then be converted to a detection flag, a localized emission region/source, and/or an emission rate, so that the leak or fugitive emission can be quantified (usually as a volumetric or mass flowrate) via a direct measurement of wind speed and direction (and associated variability—or inferred from hyperlocal forecast data site) or a simulated flow field derived through the measurement of other physical parameters.

These measurements may be undertaken on a number of spatial and temporal scales by a number of different providers. As described herein, emission, leak, and gas source may be used interchangeably. Embodiments of the disclosed integrated solution herein may employ one or more of the following: an early identification of leaks/emissions, categorization of the leaks/emissions (e.g. high/medium/low, good/bad, alarm/no alarm, etc.), localization of the leaks/emissions, which may also be on a range of length scales, particularly for remote operations; a frequency of measurement that enables users to minimize lost gas in the most cost-effective manner; and/or quantification of leak/emission rate, whether to calculate how much has been emitted, emissions for internal/external to entity reporting (e.g. ESG), and enabling carbon credits calculations.

The system and method disclosed herein covers the use of a variety of different sensors, instruments and deployment methods to optimally find, attribute, and enables the rapid fix of leaks. These systems and methods may utilize a real-time integration of emissions data into existing data management systems, which can provide additional context to the source, location and magnitude of the emissions, and may also enable abrupt limitation of aforementioned emissions with automation, a key element of regulations.

Production facilities may have one or more industrial networks or Supervisory Control and Data Acquisition Systems (SCADA) systems that may use a range of sensors (pressure, temperature, flowrate, etc.) to manage production from the various valves, pipelines, compressors, etc.

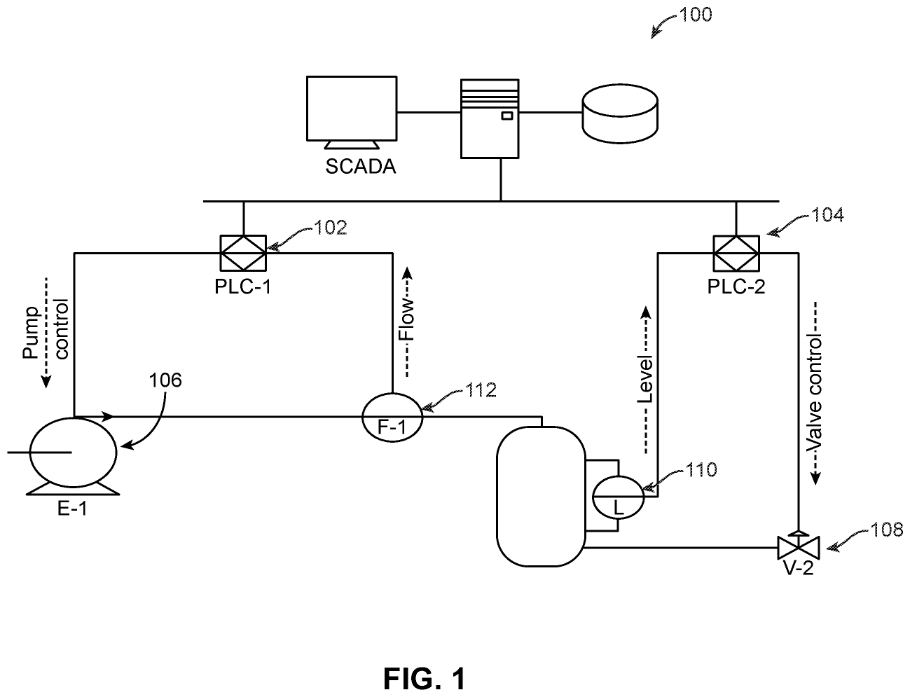

A schematic of an embodiment of an industrial network system 100 managing a production process is shown in FIG. 1, wherein the industrial network system 100 reads the measured flow 112 and flow level 110 readings which are controlled using the various connected components such as a PLC-1 102 and PLC-2 104. In some embodiments, the industrial network system 100 may be a SCADA system. In the embodiment shown in FIG. 1, PLC-1 102 compares the measured flow 112 to a previously established setpoint and controls a pump E-1 106 speed as required to match a flow level 110 to the setpoint. PLC-2 104 compares the measured flow level 110 to the setpoint and controls the flow 112 through a valve V-2 108 to match flow level to the setpoint.

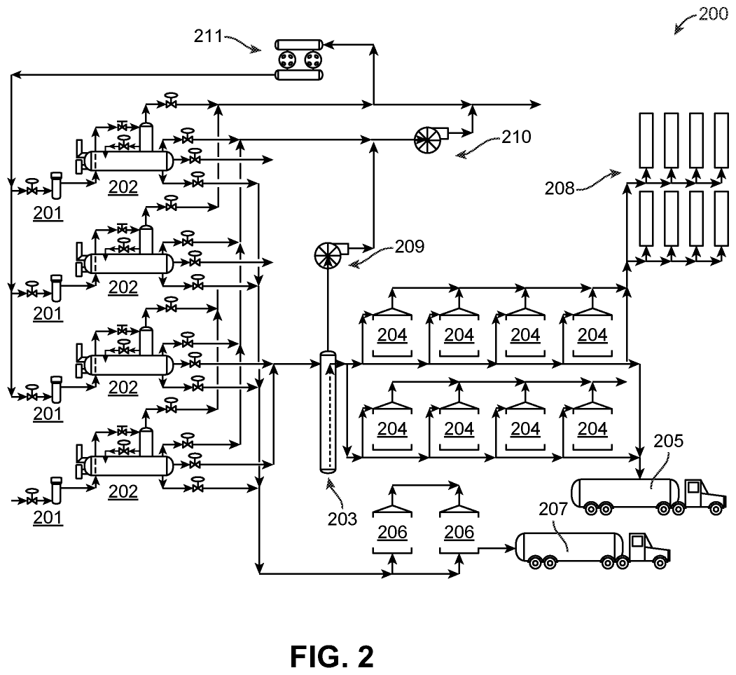

These industrial networks or SCADA systems may be used to identify potential disruptions from normal operations, including highlighting risks for leaks. There may be many degrees of complexity from the relatively simple flow-loop shown in FIG. 1, to the more complex asset or multiple well-pad management systems 200, such as that shown in FIG. 2. In the complex embodiment depicted in FIG. 2, the complex asset or multiple well-pad management system comprises additional system components which require control and monitoring. Such system components include: wellheads 201, two-stage separators with chokes 202, a vapor recovery tower 203, oil storage tanks 204, an oil hauling truck 205, water storage tanks 206, a water hauling truck 207, an emissions combustion device 208, a low-pressure vapor recovery unit 209, a low-pressure VRU 210, and a gas lift compressor 211. The systems and methods disclosed herein apply equally to all scales of systems.

Data from real-time or near-real-time SCADA measurements may be used to immediately identify particular components that may need a closer inspection. For example, a local pressure sensor may have indicated a drop in pressure between two stations that could highlight a potential leak.

In some embodiments, the disclosed system and method may include a network of things communicating, e.g., a human operator, satellite detections, after a maintenance event, after a set number of gas monitor alarms, etc. Any of these network of things communicate may trigger a localized survey. In some embodiments, priority may be given to one triggering event over another.

To this end, an aerial vehicle such as an unmanned aerial vehicle or a drone could then be triggered to go and inspect the specific location, performing careful (pre-programmed) flights around the specific equipment group (or component). The triggered device, e.g., drones, may use manual controlled cameras, automated controlled cameras, and/or any other device in addition to the gas sensor onboard. These devices may include SLAM (simultaneous localization and mapping) technologies to generate 3D maps in near real-time and leverage artificial intelligence (AI) and object recognition to attribute sources. This generation and object detection may be performed in real-time or in post processing.

In some embodiments, the triggered device may be a ground mobile robotics and/or a surface mobile robotics. In some embodiments, the inspection may not be limited to the inspection of just onshore assets and may apply to a distribution of offshore areas of interest (AOI). In some embodiments, the triggered device, e.g., drones, may be pre-programmed with various flight paths or may be flown manually as part of a response. The level of automation may vary in complexity to a perimeter flight, to individual flights around specific equipment groups. In some embodiments, the aerial vehicle may be stored in a self-contained enclosure for such pre-programmed flights. In some embodiments, the aerial vehicle is bound to a geographical region and can fly a dynamic flight pattern to localize the emission source(s) to specific equipment groups or smallest region possible.

In some embodiments, it may be preferred that the whole equipment layout/location/type and expected emissions (and an optimal flight plan around such equipment) has been built into the known flight locations of the drone (via specific waypoints or other such continuous mapping mechanism), so it can immediately know when and where to deploy once it gets the signal that the industrial network or SCADA system has seen something that could potentially be a leak. Emissions are three dimensional. In some embodiments, the drones may land and measure for a specified period of time such that the drone is treated as a mobile continuous sensor. This information may include not only location, function, equipment type etc., but more detailed information such as operating specification, bottoms-up emissions calculation, serial number, manufacturer, and links to repair history, etc. The drone may also make emissions and wind measurements as the drone gets to the desired location to refine its localization and optimization calculations. In some embodiments, an optical gas imaging (OGI) camera may be added to the drone to refine uncertainty. In some embodiments, a carbon dioxide (CO₂) sensor may be added to the drone as well, which would also give an indication of flare combustion efficiency (another source of methane). These sensors may be considered not just leak-related. In some embodiments, sensors may be added for ethane for anthropogenic vs naturally occurring discrimination; sensors may be added for other Volatile organic compounds; sensors may be added for relative humidity and other atmospheric measurements to allow for improvements to the boundary layer modeling. Publicly available models such as Methane Emissions Estimate Tool (MEET), Fugitive Emissions Abatement Simulation Toolkit (FEAST), or other proprietary model to assist in source attribution or determine emission abatement efficacy.

In one preferred embodiment, the facility may have a drone with a high-sensitivity methane sensor ready to deploy (such as a drone in an enclosure with continuous charge from a base station), and all equipment group locations already pre-programmed in In some embodiments, onboard detection methods may include taking various measurements from different points to obtain a maximum density emissions value to pinpoint possible leak locations. Some embodiments may include a post-commissioning or start-up emissions survey of a site. In some embodiments, any leaks could be covered under warranty and may be detected earlier in such a post-commissioning or start-up emissions survey of a site. Other sensors on the drone may be used to detect VOCs and ethane to give an indication of leak vs naturally occurring trace gases. Any modifications or idiosyncrasies to the location (build-up of undergrowth or addition of new equipment) may be built up over time as the drone flies regularly repeat missions for conventional monitoring, so that it is not surprised when called out for ‘emergency’ emission validation & verification and can proceed quickly and safely (without overflying any critical equipment).

If the drone-based sensor detects one or more emissions above a minimum detection threshold (and above the ambient conditions that have been identified by previous repeat surveys), then a localization and quantification analysis may be performed to enable the leaks to be triaged. The drone based sensors may include trace gas sensors, carbon dioxide sensors (CO₂), H2 sensors, Ethane sensors, ammonia sensors, SOx sensors, NOx sensors, and/or other Volatile Organic Compound (VOC) sensors. In a preferred embodiment, the emission with the highest methane leak rate would be fixed first.

Once the emission has been identified, the drone can (via its in-built telemetry system communicate directly back to the industrial network or SCADA system to indicate that flow can be shut-off or diverted, and at the same time alert a pre-selected repair group (or robotic technology) to perform the necessary repair or remedial action. In some embodiments, there may be a local LAN or IIOT network that may be used for this sensor and/or drone for communication. Once the repair has been affected and signed-off, the digital workflow system can request the industrial network or SCADA system to enable flow to the specific equipment again whilst simultaneously also requesting that the drone performs a validation of the repair.

If there are multiple emissions detected and they have been triaged, multiple repair teams in parallel, or one team in series can make the repairs; these repairs can then be validated by automatically redeploying the trace gas sensor, and production restored in a manner such that the cost function that minimizes the overall monetary cost, enabling production to be restored in an effective and timely manner.

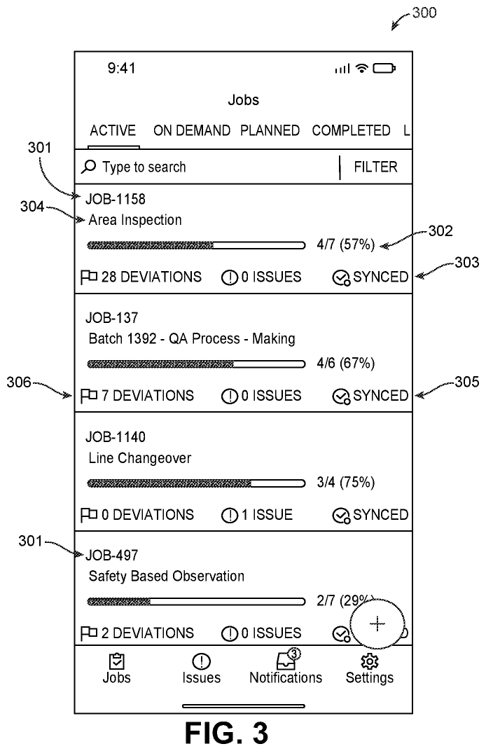

In a preferred embodiment, the whole system may be deployed via a digital workflow management system, such as that in software utilizing a graphical user interface (GUI) 300, and example of which can be seen in FIG. 3. In this preferred embodiment, the GUI 300 provides information to a user concerning on-going jobs 301 such as a completion percentage 302, a job data sync indicator 303, a job title 304, number of issues 305 related to a job, as well as the number of job deviations 306.

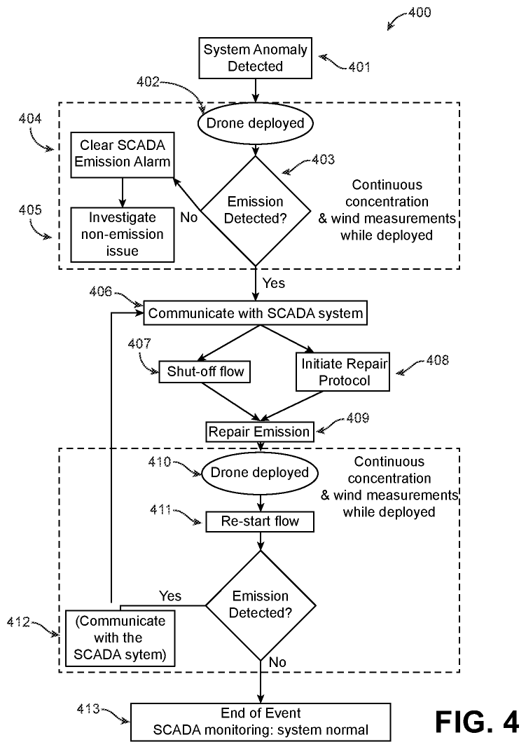

FIG. 4 depicts an overall workflow 400 for automated emission detection and repair with integrated industrial network system. The workflow 400 may comprise the system initially detecting an anomaly 401 and the deployment of a drone and/or UAV 402. If an emission is not detected, then the industrial network or SCADA emission alarm may be cleared 404 and further steps may be taken to investigate the non-emission issue 405. If an emission is detected, then the drone and/or a Ground Control System (GCS) may communicate with the industrial network or SCADA system 406. In some embodiments, flow may be shut-off 407 in response to detected emissions. In other embodiments, a repair protocol 408 may be initiated in response to detected emissions. The workflow 400 may then include initiating a repair 409 to stop the emissions.

The workflow 400 may then include deploying a drone or UAV 410. In some embodiments, this may be the same drone or UAV used once the system anomaly was detected. In some embodiments, the drone or UAV may be contained in a housing or base station. The flow may then be re-started 411 if the flow was stopped. The drone or UAV may continuously collect concentration and wind measurements while deployed. If another emission is detected, then the drone or UAV or GCS may communicate with the industrial network or SCADA system 412. If another emission is not detected, then the system may determine the system is normal and end the event 413.

Measurements may be undertaken on a number of spatial and temporal scales by a number of different providers. An integrated solution may include one or more of the following: early identification of leaks (both ‘small’ and ‘large’); localization of the leaks, this can also be on a range of length scales, particularly for remote operations; a frequency of measurement that enables minimizing lost gas in the most cost-effective manner; and/or quantification of leak rate, whether to calculate how much has been lost to enabling carbon credits calculations.

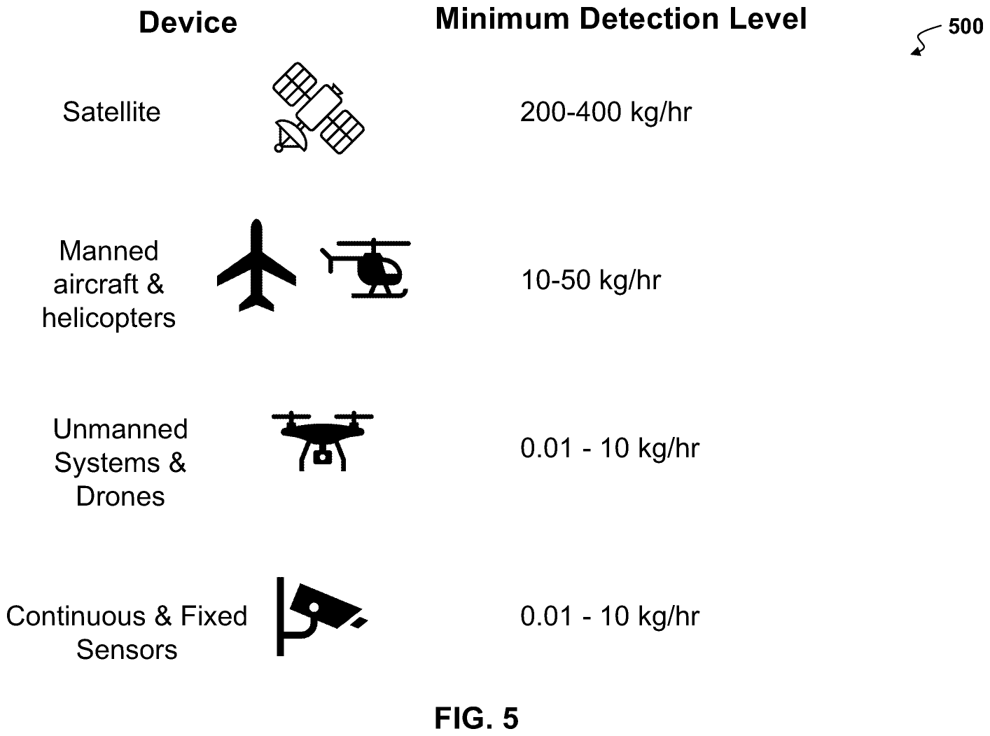

FIG. 5 depicts differing scales for emissions monitoring 500. All of these factors may operate at different length, time and cost scales. As a result, there may not be a one size fits all solution, as shown in FIG. 5. The disclosed system and method allows for effectively using the range of available measurements, artificial intelligence, and historical information to optimize the coverage for a particular customer or user who may have multiple assets and operations at various stages of development and accessibility. The different devices have different sensitivities for detecting trace gas. A satellite may have a minimum detection level of 200-400 kg/hr. A manned aircraft and/or helicopter may have a minimum detection level of 10-50 kg/hr. An unmanned system and/or drone may have a minimum detection level of 0.01-10 kg/hr. A continuous and fixed sensor may have a minimum detection level of 0.01-10 kg/hr.

The disclosed system and method takes the role of an integrated service provider, providing the optimum solution for the customer or user depending upon their monitoring needs, and how those also relate to the relevant regulatory framework at that time. In this case, the disclosed system and method can identify a number of scenarios which can optimize the emissions leak detection (and ultimately) repair, by smart identification.

The disclosed method may comprise the following two items. First, the disclosed method may use a regular use of a satellite (or constellation of satellites) if available for weekly or bi-weekly monitoring of all operations (if there is a constellation of satellites from one or more providers). Polar orbiting satellites, such as those provided by GHGSat can effectively cover almost every point on the planet. In some embodiments, these polar orbiting satellites may not effectively detect leaks over water although due to their reflectivity-based spectroscopy method. For land-based operations, the disclose system and method may use periodic flyovers that could be undertaken every few days (with a fully populated).

If a leak is spotted, this would trigger one (or more of the following): The Use of an airborne survey (using equipment such as that provided by companies such as Kairos, the airborne sensor version of that provided by GHGSat, Scientific Aviation, etc., for a broad areal coverage, and possible high level or large emissions quantification) and/or the deployment of a drone-based high-resolution/high sensitivity survey for the particular site identified by the coarser measurement that targets the specific equipment group and then provides localization and quantification.

This triggering of the more localized, higher accuracy survey would be automated, based on a pre-determined (or adaptable) threshold, with safeguards to ensure no false alarming.

Second, the disclosed method may use a pre-existing fixed, mesh-sensor network (Industrial internet of things-IIoT) that is based on lower resolution sensors to trigger a more in-depth drone analysis if it detects a leak above a predetermined threshold of detection.

The drone-based survey may then use the triangulation of the mesh sensor network to home in on the specific equipment group or component of interest and then perform a complete survey. This may also trigger a callout for a remote or locally contracted repair team to effect a fix to the leak or fix the leaks in the order of importance if multiple leaks are identified and triaged by the system (typically as a function of leak size or severity of impact to ongoing operations and safety).

This would be in a manner similar to that for a smoke detector in a house triggering a small house-based robot to go and investigate where the source of the smoke might be and identify the nature of the source/fire depending on the room in which the smoke alarm is based.

Alternative embodiments of the above could also include the drone survey triggering either an optical gas imaging camera or a handheld version of a trace gas high sensitivity sensor, both of which could be deployed by a person, or optimally by drone or surface-based robotic system.

An alternative scenario is to use a semi-fixed mesh sensor network that can reconfigure itself based on specifically identified leaks.

In some embodiments, the system may include one or more manned aircraft surveys.

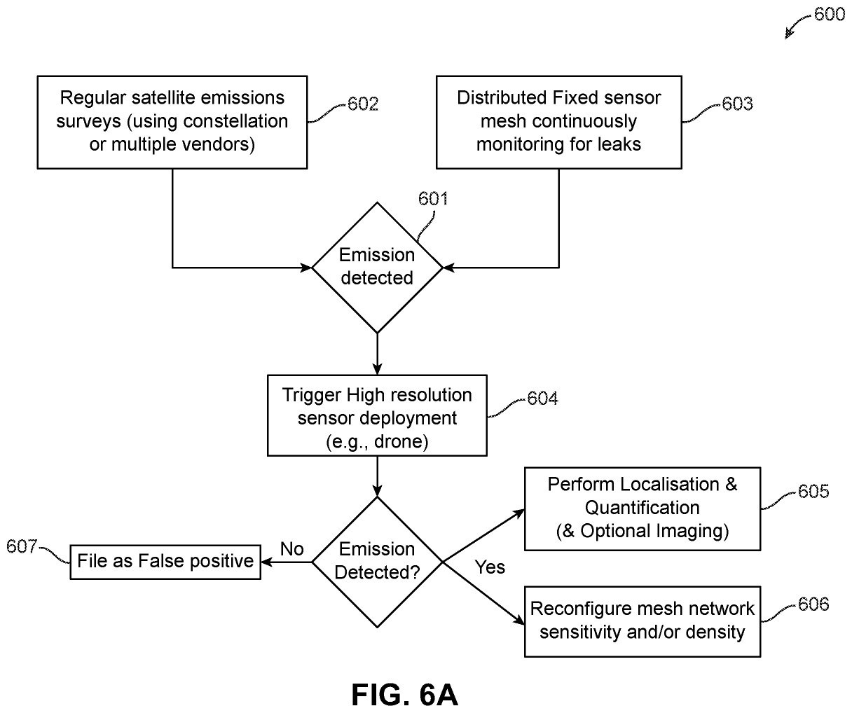

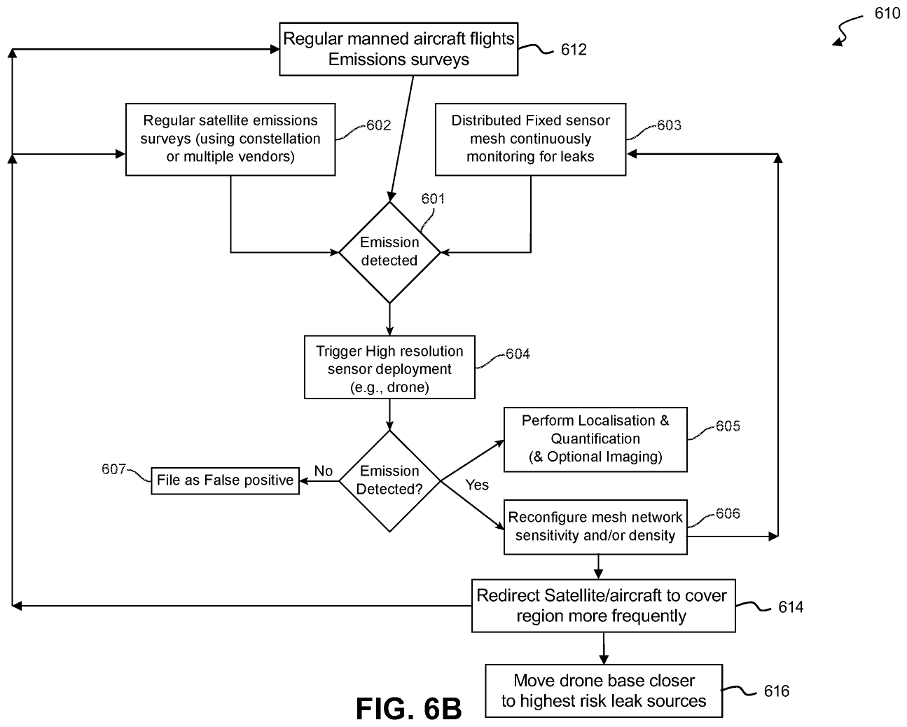

The flow chart 600 shown in FIG. 6A below highlights an embodiment of these events and decisions. An emission may be detected 601 by a regular satellite emissions survey 602, or a distributed fixed sensor mesh, a manned aircraft with one or more sensors, and/or a helicopter with one or more sensors continually monitoring for leaks 603. Once an emission is detected 601, a high-resolution sensor may be deployed 604. In some embodiments, this sensor may be deployed on a drone, such as an aerial vehicle or an unmanned aerial vehicle (UAV). If an emission is still detected, the system may perform localization and quantification (and optional imaging) 605 and/or reconfigure the mesh network sensitivity and/or density 606. If an emission is not detected, the system may file the detected emission as a false positive 607.

Another alternative embodiment is to use a hybrid of approaches combining embodiments above, whereby the satellite emissions 602 could reconfigure the mesh sensor network 603 and that in turn would check for a leak above a given minimum detection threshold and trigger the deployment of a more accurate sensor method, e.g., the drone-based sensor or a reconfiguration of the mesh sensor network to build more redundancy into the system.

A drone in a housing of a base station may also function as one type of networked fixed sensor.

The disclosed system and method may be used to build a database that may be used to highlight the following: those geographical regions that have higher than average leaks/emissions; equipment groups that have higher than average leaks/emissions; equipment components that have higher than average leaks/emissions; and/or equipment OEMs that have higher than average leaks/emissions

A machine-learning algorithm may then be used on global data to look at predictive maintenance based on the statistical information to minimize future emissions.

The use of artificial intelligence may be used to determine those equipment groups or areas that will be more likely to emit trace gasses. The disclose system and method may use this information on likelihood to ensure that the satellites are targeted more frequently over those locations, and that the drone flights are undertaken more frequently around those specific equipment groups. Similarly, the false positive information triggered by either the satellite or local mesh network may also go towards optimizing the revisit frequency and density and sensitivity of future measurements in those regions.

Similarly, the information seen from sparse surveys can also be used to trigger more dense surveys in regions where there are elevated emissions concentrations above background.

In addition to satellite and fixed sensor triggering higher resolution and sensitivity localization and quantification (e.g., drone-deployed trace gas sensors), the disclosed system and method may utilize (manned) aircraft surveys also highlighting areas for the more in-depth surveys in some embodiments.

Additionally, optical gas imaging cameras (either mobile, or semi-fixed) or alternative fixed sensors (laser/LIDAR) may also be used to trigger events that need quantification, and this may be used to re-direct the regular drone surveys off their standard waypoints to perform a more dense set of concentration measurements around a specific set of equipment or equipment group. This is a version of dynamic re-tasking may be used to conduct highly efficient and cost-effective surveys.

FIG. 6B depicts a workflow 610 for using a closed loop feedback to probability of detection in emissions monitoring and reduction, according to one embodiment. FIG. 6B depicts how the drone may be moved, or the satellites, aircraft, and even continuous measurements may all be redeployed to focus on other regions hence optimizing the possibility of detection and assisting in the refinement of emission rate uncertainty. The steps in FIG. 6B include the steps described in FIG. 6A with a feedback loop added. The workflow 610 may include regular manned aircraft flights and/or emission surveys 612. Emissions may be detected 601 based on these include regular manned aircraft flights and/or emission surveys 612 in addition to regular satellite emissions surveys 602 and/or distributed fixed sensor mesh that is continuously monitoring for trace gas leaks 603. If an emission is detected and the mesh network sensitivity and/or density is reconfigured 606, a satellite and/or an aircraft may be redirected to cover the region where the emission was detected more frequently 614. The satellite and/or an aircraft that may be redirected to cover the region where the emission was detected more frequently 614 may impact the regular satellite emissions surveys 602, the distributed fixed sensor mesh 603, and/or the regular manned aircraft flights 612. A drone base may be moved closer to the highest risk leak sources 616.

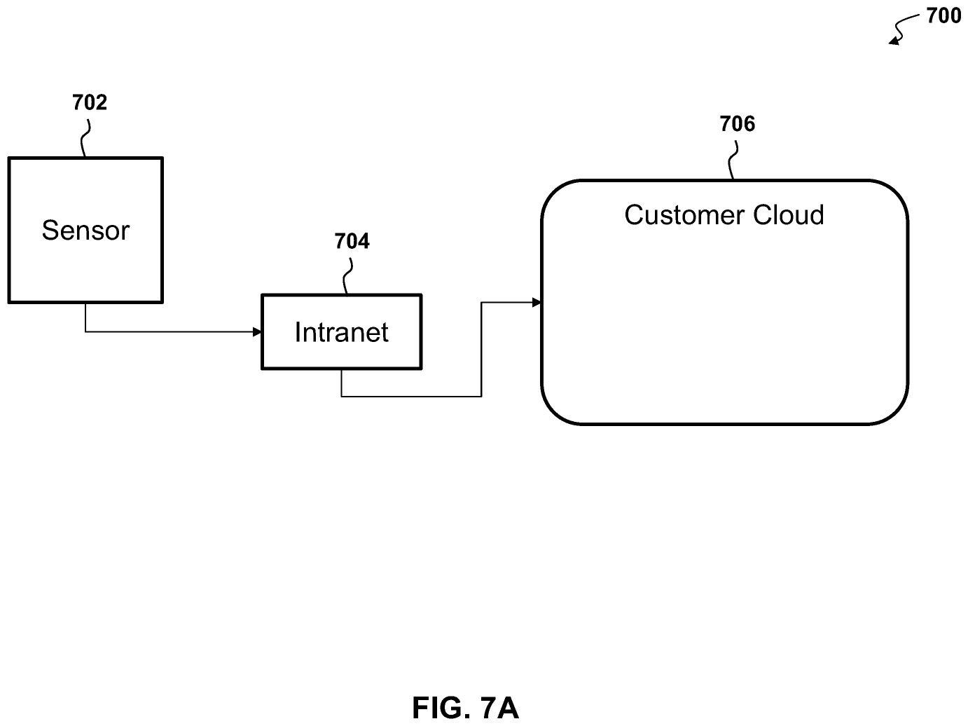

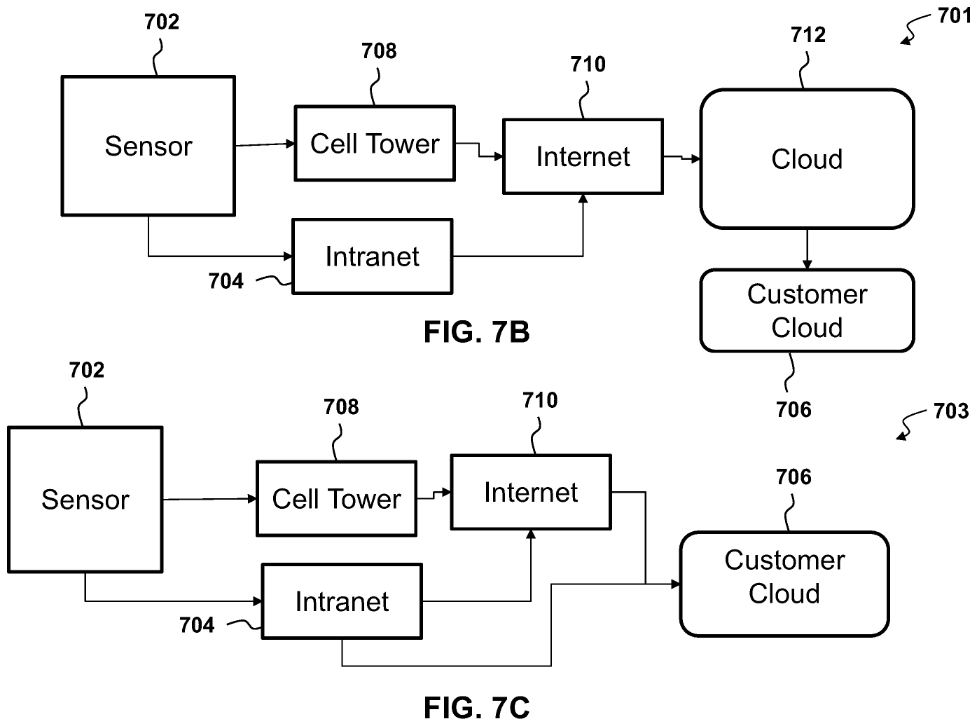

FIGS. 7A-7C depict various communication systems for communications between one or more sensors and a cloud server. FIG. 7A depicts a communication system 700. The communications system 700 may include one or more sensors 702, such as trace gas sensors, in communication with an intranet 704. The intranet 704 may then be in communication with a customer cloud server 706. FIG. 7B depicts an alternate communication system 701 for a first scenario. The alternate communication system 701 may include one or more sensors 702 in communication with a cell tower 708 and/or an intranet 704. The cell tower 708 and/or intranet 704 may be in communication with the Internet 710. The Internet 710 may be in communication with the cloud server 712. The cloud server 712 may be in communication with a customer cloud 706. FIG. 7B depicts an alternate communication system 703 for a second scenario. The alternate communication system 703 may include one or more sensors 702 in communication with a cell tower 708 and/or an intranet 704. The cell tower may be in communication with the Internet 710. The Internet 710 may be in communication with a customer cloud server 706. The intranet 704 may be in communication with the Internet 710 and/or the customer cloud 706. The disclosed communication systems 700, 701, 703 may provide communication of data from the one or more sensors 702 to the customer cloud server 706 for storage and/or further processing.

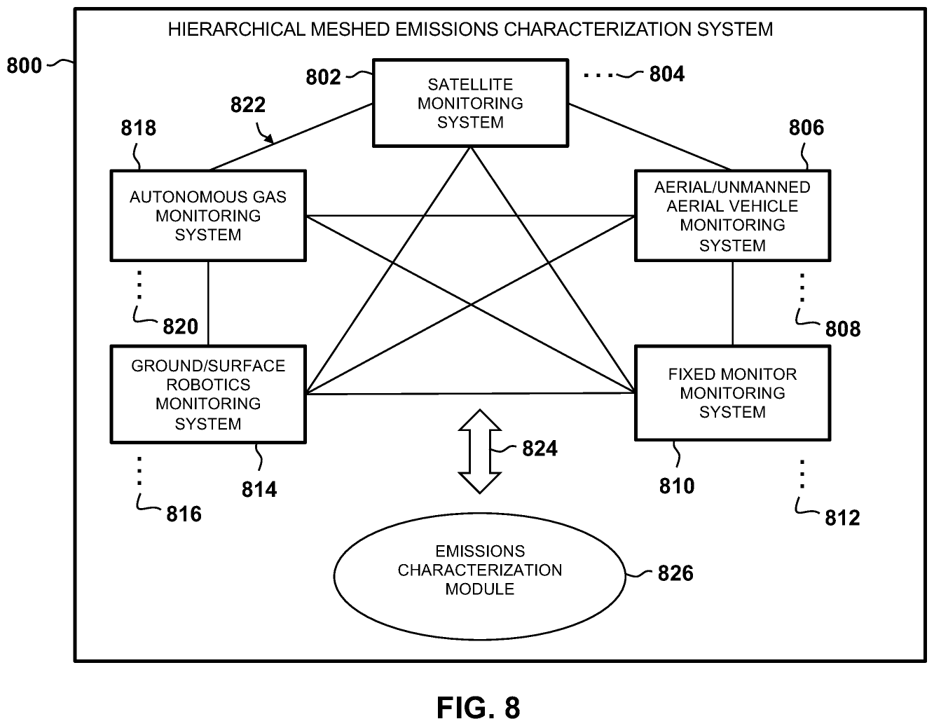

FIG. 8 depicts a hierarchical meshed emissions characterization system 800, according to one embodiment. The hierarchical meshed emissions characterization system 800 may include a mesh 822 comprising wired and/or wireless connections between one or more satellite monitoring systems 802, 804; one or more aerial and/or unmanned aerial vehicle (UAV) monitoring systems 806, 808; one or more fixed monitor monitoring systems 810, 812; one or more ground and/or surface robotics monitoring systems 814, 816; and/or one or more autonomous gas monitoring systems 818, 820. The plurality of dots 804, 808, 812, 816, 820 indicate additional monitoring systems that may be included in some system 800 embodiments. One or more trace gas emission monitoring devices (802, 804, 806, 808, 810, 812, 814, 816, 818, 820) may be devices in some embodiments. One or more trace gas emission monitoring devices (802, 804, 806, 808, 810, 812, 814, 816, 818, 820) may be systems in other embodiments such as systems including a processor with addressable memory and a trace gas sensor in communication with the processor for measuring trace gas levels, detecting trace gas amounts and/or anomalous trace gas emissions, and/or monitoring trace gas levels and/or anomalous trace gas emissions. These systems may, alone or in communication with one another, perform one or more of: alert, verify, and/or quantify anomalous trace gas emissions. The one or more trace gas emission monitoring devices (802, 804, 806, 808, 810, 812, 814, 816, 818, 820) may be connected to one another via a wired and/or wireless connection via respective wired and/or wireless network communication interfaces, transmitters, receiver, transceivers, and/or radios.

The monitoring systems 802, 804, 806, 808, 810, 812, 814, 816, 818, 820 may generate data 824 relating to trace gas measuring, detecting, and/or monitoring. The monitoring systems 802, 804, 806, 808, 810, 812, 814, 816, 818, 820 may each perform one or more of: alert, verify, and quantify anomalous trace gas emissions. This generated data 824 may be used to detect anomalous emissions of trace gasses.

The satellite monitoring system 802, 804 may generate data from one or more satellites. The one or more satellites may have low leak rate resolution, but very large area coverage. A constellation of satellites of two or more satellites may allow the satellite monitoring system 802, 804 to revisit of a particular location every few days. The satellite monitoring system 802, 804 may be used to detect a large concentration of trace gasses, such as from a significant leak of trace gasses from equipment in a location. The satellite monitoring system 802, 804 may have a minimum detection level of 200-400 kg/hr of trace gas. In some embodiments, satellite monitoring system 802, 804 may generate data on trace gas concentrations via the reflectivity-based spectroscopy method.

The aerial and/or unmanned aerial vehicle (UAV) monitoring system 806, 808 may utilize one or more of: manned aerial vehicles, helicopters, and UAVs with sensors disposed therein. The sensors on the aerial and/or UAV monitoring system 806, 808 may have a very low minimum detection level of trace gasses, such as 0.01-10 kg/hr. In some embodiments, the aerial and/or UAV monitoring system 806, 808 may only be deployed after an anomalous emission is detected by one of the other monitoring systems 802, 804, 810, 812, 814, 816, 818, 820. If an emission is still detected after deployment of the aerial and/or UAV monitoring system 806, 808, the system 800 may perform localization, quantification, optional imaging, and/or reconfigure the mesh 822 sensitivity and/or density.

The fixed monitor monitoring system 810, 812 may include one or more sensors fixed or semi-fixed in a location. The fixed monitor monitoring system 810, 812 may include sensors for laser and/or LIDAR. The fixed monitor monitoring system 810, 812 may include sensors with a minimum detection level of 0.01-10 kg/hr for trace gasses. In some embodiments, the fixed monitor monitoring system 810, 812 may include sensors with a lower resolution and in a greater quantity so potential anomalous emissions of trace gasses can be detected and confirmed with other monitoring systems, such as a highly accurate sensor in the aerial and/or UAV monitoring system 806, 808.

The ground and/or surface robotics monitoring system 814 may include one or more robots configured to move about on the ground such as via one or more wheels, treads, legs, or the like. The ground and/or surface robotics monitoring system 814 may allow for movement of sensors disposed therein so as to determine potential anomalous emissions of trace gasses. For example, a ground robot with a trace gas sensor may be moved towards a potential trace gas leak of equipment downwind of the potential trace gas leak. An increase in trace gas measurements as the ground robot moves toward the equipment could indicate a higher likelihood that the trace gas leak origin is the equipment. Similarly, a decrease in trace gas measurements as the ground robot moves toward the equipment could indicate a lower likelihood that the trace gas leak origin is the equipment. As another example, a cluster of ground robots could be moved to a location of a potential trace gas leak to determine if the change in trace gas measurements from trace gas sensors located on the ground robots indicates a higher or lower likelihood of a trace gas leak from the location in conjunction with data from the other monitoring systems 802, 804, 806, 808, 810, 812, 818, 820.

The autonomous gas monitoring system 818, 820 may include one or more autonomous systems with trace gas sensors for generating data for potential anomalous emissions of trace gasses.

Data 824 from the mesh may be sent to an emissions characterization module 826. In some embodiments, the emissions characterization module 826 may alert, verify, and quantify anomalous emissions activities. In other embodiments, the monitoring systems 802, 804, 806, 808, 810, 812, 814, 816, 818, 820 in the mesh 822 may work in concert to alert, verify, and quantify anomalous emissions activities. The monitoring systems 802, 804, 806, 808, 810, 812, 814, 816, 818, 820 in the mesh 822 may take certain actions based on an anomalous emissions activities such as elevated trace gas reading to determine if the anomalous emissions activities are caused by a trace gas leak, such as via faulty equipment, or caused by a false positive. If the anomalous emissions activities are the result of a trace gas leak, the system 800 may determine that corrective actions need to be taken, such as a repair of equipment. The determination that anomalous emissions activities are the result of a trace gas leak may be used in a feedback loop of the data 824 to ensure more accurate characterizations of anomalous emissions activities in the future. Similarly, the determination that anomalous emissions activities are the result of a false positive may be used in a feedback loop of the data 824 to ensure more accurate characterizations of anomalous emissions activities in the future and reduce the likelihood of future false positives when similar data 824 is generated by the monitoring systems 802, 804, 806, 808, 810, 812, 814, 816, 818, 820.

In one embodiment, the emissions characterization module 826 comprises a centralized computing system that implements the method disclosed herein as instructions executed by a processor. In another embodiment, the function of the emissions characterization module 826 is implemented in a distributed fashion such as performed by processors and memory in one or more of the monitoring systems 802, 804, 806, 808, 810, 812, 814, 816, 818, 820 in the mesh network 822. In other embodiments of the system and method of FIG. 8, other network topologies for communication may also be implemented (e.g., star, bus, ring, hybrid, etc).

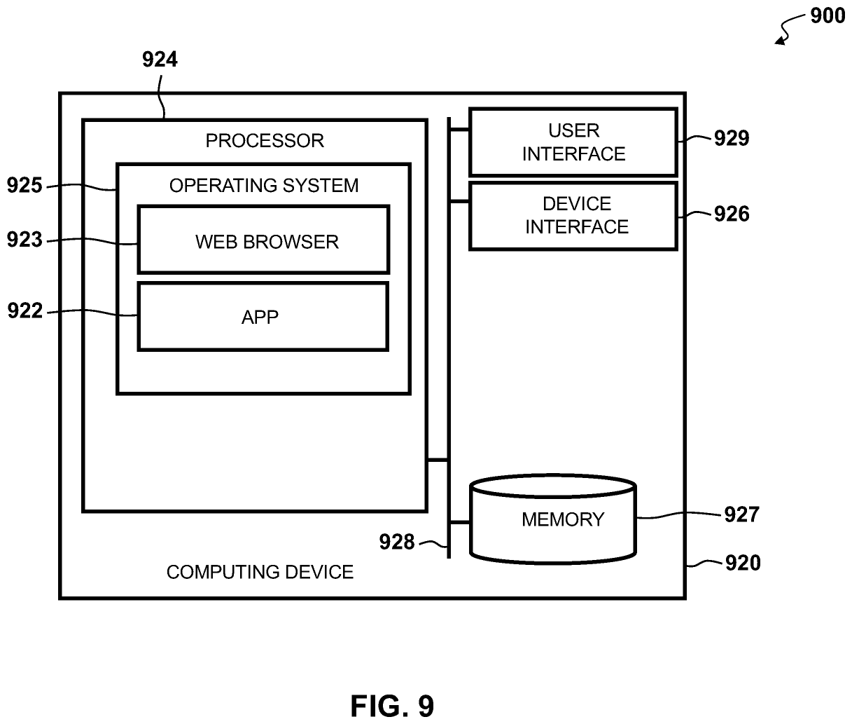

FIG. 9 illustrates an example of a top-level functional block diagram of a computing device embodiment 900. The example operating environment is shown as a computing device 920 comprising a processor 924, such as a central processing unit (CPU), addressable memory 927, an external device interface 926, e.g., an optional universal serial bus port and related processing, and/or an Ethernet port and related processing, and an optional user interface 929, e.g., an array of status lights and one or more toggle switches, and/or a display, and/or a keyboard and/or a pointer-mouse system and/or a touch screen. Optionally, the addressable memory may, for example, be: flash memory, eprom, and/or a disk drive or other hard drive. These elements may be in communication with one another via a data bus 928. In some embodiments, via an operating system 925 such as one supporting a web browser 923 and applications 922, the processor 924 may be configured to execute steps of a process establishing a communication channel and processing according to the embodiments described above.

System embodiments include computing devices such as a server computing device, a buyer computing device, and a seller computing device, each comprising a processor and addressable memory and in electronic communication with each other. The embodiments provide a server computing device that may be configured to: register one or more buyer computing devices and associate each buyer computing device with a buyer profile; register one or more seller computing devices and associate each seller computing device with a seller profile; determine search results of one or more registered buyer computing devices matching one or more buyer criteria via a seller search component. The service computing device may then transmit a message from the registered seller computing device to a registered buyer computing device from the determined search results and provide access to the registered buyer computing device of a property from the one or more properties of the registered seller via a remote access component based on the transmitted message and the associated buyer computing device; and track movement of the registered buyer computing device in the accessed property via a viewer tracking component. Accordingly, the system may facilitate the tracking of buyers by the system and sellers once they are on the property and aid in the seller's search for finding buyers for their property. The figures described below provide more details about the implementation of the devices and how they may interact with each other using the disclosed technology.

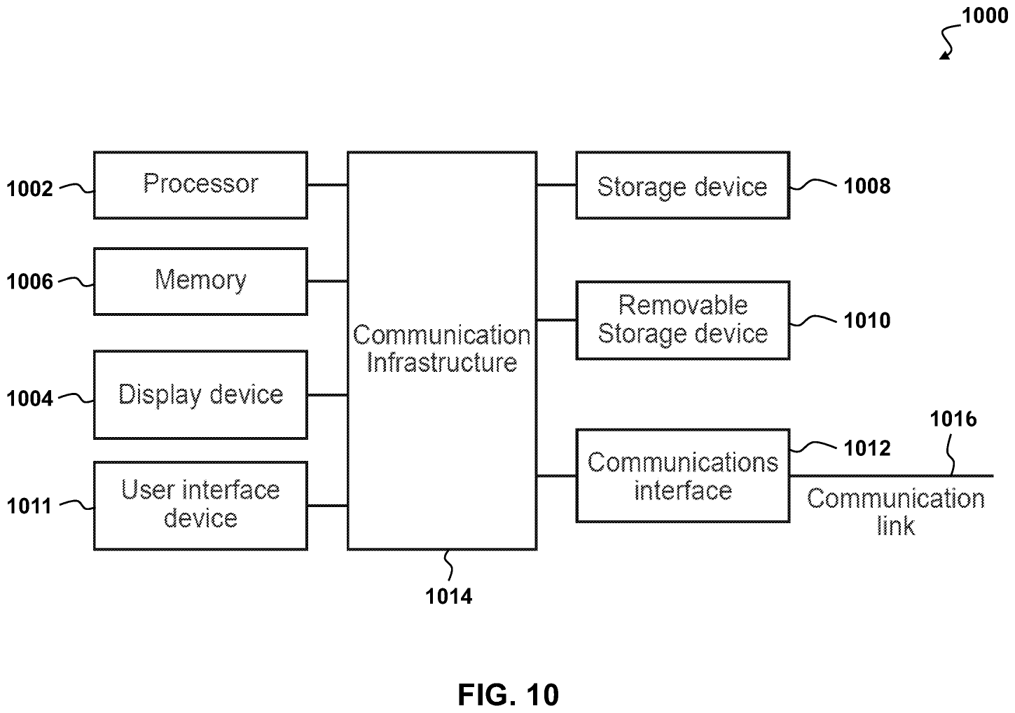

FIG. 10 is a high-level block diagram 1000 showing a computing system comprising a computer system useful for implementing an embodiment of the system and process, disclosed herein. Embodiments of the system may be implemented in different computing environments. The computer system includes one or more processors 1002, and can further include an electronic display device 1004 (e.g., for displaying graphics, text, and other data), a main memory 1006 (e.g., random access memory (RAM)), storage device 1008, a removable storage device 1010 (e.g., removable storage drive, a removable memory module, a magnetic tape drive, an optical disk drive, a computer readable medium having stored therein computer software and/or data), user interface device 1011 (e.g., keyboard, touch screen, keypad, pointing device), and a communication interface 1012 (e.g., modem, a network interface (such as an Ethernet card), a communications port, or a PCMCIA slot and card). The communication interface 1012 allows software and data to be transferred between the computer system and external devices. The system further includes a communications infrastructure 1014 (e.g., a communications bus, cross-over bar, or network) to which the aforementioned devices/modules are connected as shown.

Information transferred via communications interface 1014 may be in the form of signals such as electronic, electromagnetic, optical, or other signals capable of being received by communications interface 1014, via a communication link 1016 that carries signals and may be implemented using wire or cable, fiber optics, a phone line, a cellular/mobile phone link, an radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface 1012. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

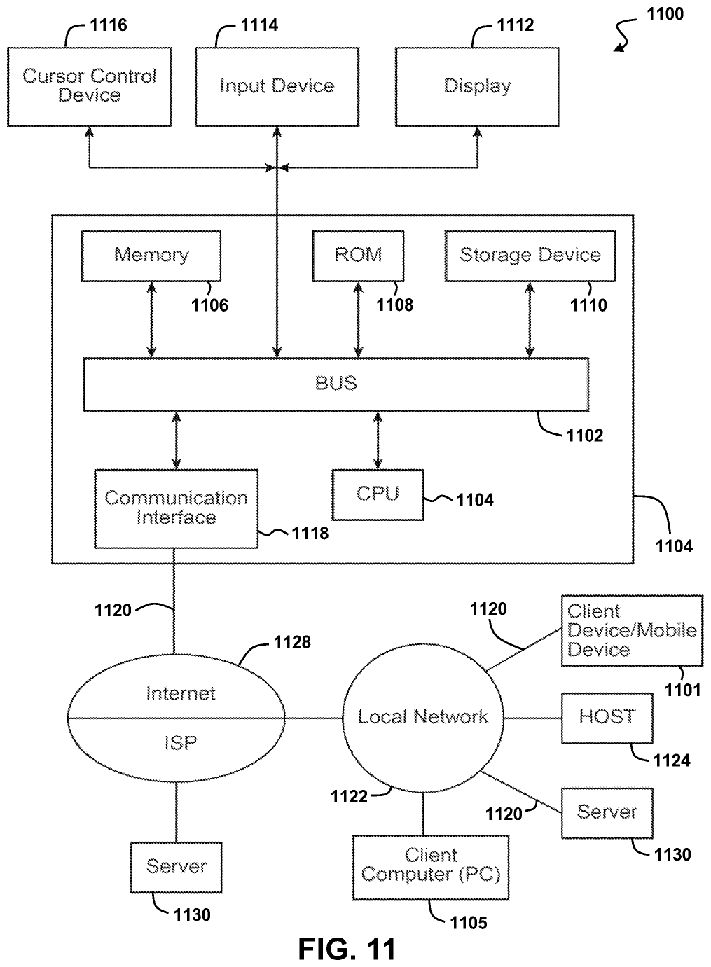

FIG. 11 shows a block diagram of an example system 1100 in which an embodiment may be implemented. The system 1100 includes one or more client devices 1101 such as consumer electronics devices, connected to one or more server computing systems 1130. A server 1130 includes a bus 1102 or other communication mechanism for communicating information, and a processor (CPU) 1104 coupled with the bus 1102 for processing information. The server 1130 also includes a main memory 1106, such as a random access memory (RAM) or other dynamic storage device, coupled to the bus 1102 for storing information and instructions to be executed by the processor 1104. The main memory 1106 also may be used for storing temporary variables or other intermediate information during execution or instructions to be executed by the processor 1104. The server computer system 1130 further includes a read only memory (ROM) 1108 or other static storage device coupled to the bus 1102 for storing static information and instructions for the processor 1104. A storage device 1110, such as a magnetic disk or optical disk, is provided and coupled to the bus 1102 for storing information and instructions. The bus 1102 may contain, for example, thirty-two address lines for addressing video memory or main memory 1106. The bus 1102 can also include, for example, a 32-bit data bus for transferring data between and among the components, such as the CPU 1104, the main memory 1106, video memory and the storage 1110. Alternatively, multiplex data/address lines may be used instead of separate data and address lines.

The server 1130 may be coupled via the bus 1102 to a display 1112 for displaying information to a computer user. An input device 1114, including alphanumeric and other keys, is coupled to the bus 1102 for communicating information and command selections to the processor 1104. Another type or user input device comprises cursor control 1116, such as a mouse, a trackball, or cursor direction keys for communicating direction information and command selections to the processor 1104 and for controlling cursor movement on the display 1112.

According to one embodiment, the functions are performed by the processor 1104 executing one or more sequences of one or more instructions contained in the main memory 1106. Such instructions may be read into the main memory 1106 from another computer-readable medium, such as the storage device 1110. Execution of the sequences of instructions contained in the main memory 1106 causes the processor 1104 to perform the process steps described herein. One or more processors in a multi-processing arrangement may also be employed to execute the sequences of instructions contained in the main memory 1106. In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, 23ncludeng a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 1104 for execution. Such a medium may take many forms, including but not limited to, non-volatile media, volatile media, and transmission media. Non-volatile media includes, for example, optical or magnetic disks, such as the storage device 1110. Volatile media includes dynamic memory, such as the main memory 1106. Transmission media includes coaxial cables, copper wire and fiber optics, including the wires that comprise the bus 1102. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 1104 for execution. For example, the instructions may initially be carried on a magnetic disk of a remote computer. The remote computer can load the instructions into its dynamic memory and send the instructions over a telephone line using a modem. A modem local to the server 1130 can receive the data on the telephone line and use an infrared transmitter to convert the data to an infrared signal. An infrared detector coupled to the bus 1102 can receive the data carried in the infrared signal and place the data on the bus 1102. The bus 1102 carries the data to the main memory 1106, from which the processor 1104 retrieves and executes the instructions. The instructions received from the main memory 1106 may optionally be stored on the storage device 1110 either before or after execution by the processor 1104.

The server 1130 also includes a communication interface 1118 coupled to the bus 1102. The communication interface 1118 provides a two-way data communication coupling to a network link 1120 that is connected to the world wide packet data communication network now commonly referred to as the Internet 1128. The Internet 1128 uses electrical, electromagnetic or optical signals that carry digital data streams. The signals through the various networks and the signals on the network link 1120 and through the communication interface 1118, which carry the digital data to and from the server 1130, are exemplary forms or carrier waves transporting the information.

In another embodiment of the server 1130, interface 1118 is connected to a network 1122 via a communication link 1120. For example, the communication interface 1118 may be an integrated services digital network (ISDN) card or a modem to provide a data communication connection to a corresponding type of telephone line, which can comprise part of the network link 1120. As another example, the communication interface 1118 may be a local area network (LAN) card to provide a data communication connection to a compatible LAN. Wireless links may also be implemented. In any such implementation, the communication interface 1118 sends and receives electrical electromagnetic or optical signals that carry digital data streams representing various types of information.

The network link 1120 typically provides data communication through one or more networks to other data devices. For example, the network link 1120 may provide a connection through the local network 1122 to a host computer 1124 or to data equipment operated by an Internet Service Provider (ISP). The ISP in turn provides data communication services through the Internet 1128. The local network 1122 and the Internet 1128 both use electrical, electromagnetic or optical signals that carry digital data streams. The signals through the various networks and the signals on the network link 1120 and through the communication interface 1118, which carry the digital data to and from the server 1130, are exemplary forms or carrier waves transporting the information.

The server 1130 can send/receive messages and data, including e-mail, program code, through the network, the network link 1120 and the communication interface 1118. Further, the communication interface 1118 can comprise a USB/Tuner and the network link 1120 may be an antenna or cable for connecting the server 1130 to a cable provider, satellite provider or other terrestrial transmission system for receiving messages, data and program code from another source.

The example versions of the embodiments described herein may be implemented as logical operations in a distributed processing system such as the system 1100 including the servers 1130. The logical operations of the embodiments may be implemented as a sequence of steps executing in the server 1130, and as interconnected machine modules within the system 1100. The implementation is a matter of choice and can depend on performance of the system 1100 implementing the embodiments. As such, the logical operations constituting said example versions of the embodiments are referred to for e.g., as operations, steps or modules.

Similar to a server 1130 described above, a client device 1101 can include a processor, memory, storage device, display, input device and communication interface (e.g., e-mail interface) for connecting the client device to the Internet 1128, the ISP, or LAN 1122, for communication with the servers 1130.

The system 1100 can further include computers (e.g., personal computers, computing nodes) 1105 operating in the same manner as client devices 1101, where a user can utilize one or more computers 1105 to manage data in the server 1130.

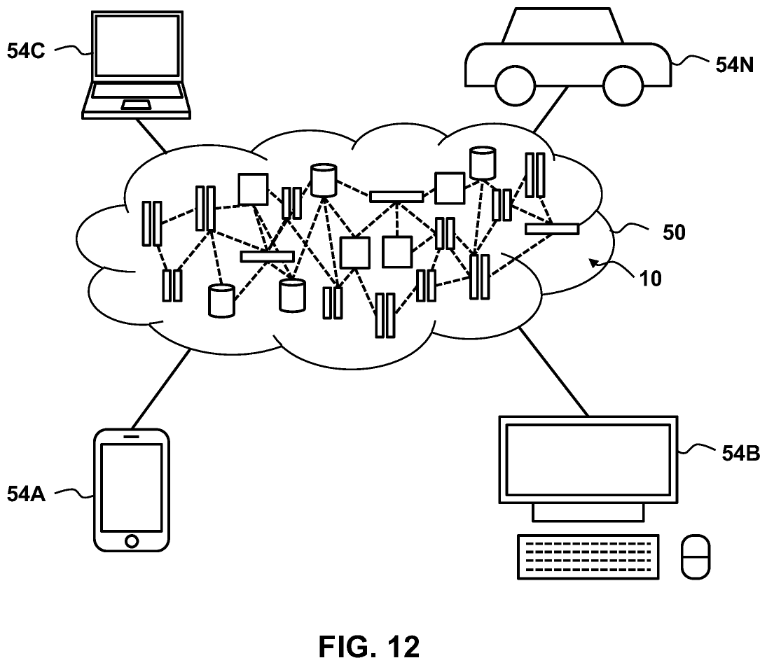

Referring now to FIG. 12, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 54B, laptop computer 54C, and/or UAV system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 12 are intended to be illustrative only and that computing nodes 10 and cloud computing environment 50 can communicate with any type of computerized device over any type of network and/or network addressable connection (e.g., using a web browser).

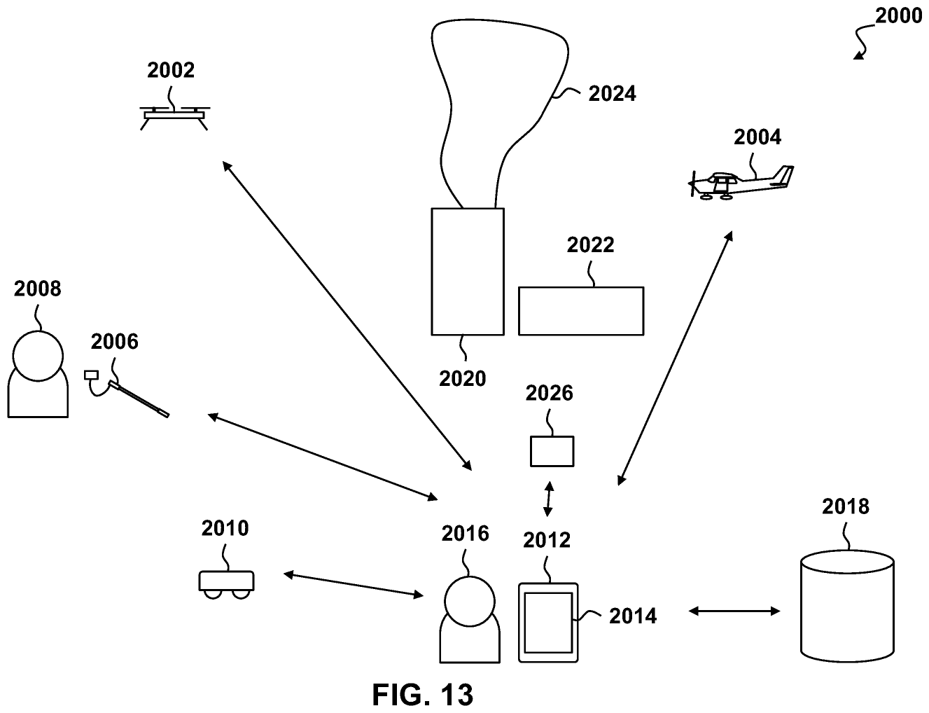

FIG. 13 depicts a system 2000 for detecting trace gasses, according to one embodiment. The system may include one or more trace gas sensors located in one or more detection vehicles 2002, 2004, 2006, 2010. The one or more trace gas sensors may detect elevated trace gas concentrations from one or more potential gas sources 2020, 2022, such as a holding tank, pipeline, or the like. The potential gas sources 2020, 2022 may be part of a large facility, a small facility, or any location. The potential gas sources 2020, 2022 may be clustered and/or disposed distal from one another. The one or more trace gas sensors may be used to detect and quantify leaks of toxic gases, e.g., hydrogen disulfide, or environmentally damaging gases, e.g., methane, sulfur dioxide) in a variety of industrial and environmental contexts. Detection and quantification of these leaks are of interest to a variety of industrial operations, such as oil and gas, chemical production, and painting. Detection and quantification of leaks is also of value to environmental regulators for assessing compliance and for mitigating environmental and safety risks. In some embodiments, the at least one trace gas sensor attached to the one or more detection vehicles 2002 2004, 2006, and 2010 may be configured to detect, for example, methane. In other embodiments, the at least one trace gas sensor may be configured to detect sulfur oxide, such as SO, SO2, SO3, S7O2, S6O2, S2O2, and the like. A trace gas leak 2024 may be present in a potential gas source 2020. The one or more trace gas sensors may be used to identify the trace gas leak 2024 and/or the source 2020 of the trace gas leak 2024 so that corrective action may be taken.

The one or more vehicles 2002, 2004, 2006, 2010 may include an unmanned aerial vehicle (UAV) 2002, an aerial vehicle 2004, a handheld device 2006, and a ground vehicle 2010. In some embodiments, the UAV 2002 may be a quadcopter or other device capable of hovering, making sharp turns, landing at measurement locations, and the like. In other embodiments, the UAV 2002 may be a winged aerial vehicle capable of extended flight time between missions. In other embodiments, the UAV 2002 may be a hybrid Vertical Takeoff and Landing (VTOL) vehicle capable of multiple methods of taking off and landing including hovering or a traditional fixed wing landing. The UAV 2002 may be autonomous or semi-autonomous in some embodiments. In other embodiments, the UAV 2002 may be manually controlled by a user. The aerial vehicle 2004 may be a manned vehicle in some embodiments. In other embodiments, the UAV 2002 may be capable of carrying additional detection devices deployable from the UAV. For example, the UAV 2002 may be deployed to a general location where several other devices capable of flight are deployed from the UAV 2002. These one or more devices may comprise measurement devices of different capabilities or the same. The handheld device 2006 may be any device having one or more trace gas sensors operated by a sensor operator 2008. In one embodiment, the handheld device 2006 may have an extension for keeping the one or more trace gas sensors at a distance from the sensor operator 2008. In other embodiments, the handheld device 2006 may have the capability of flight and/or be a hand-launched UAV 2002. The ground vehicle 2010 may have wheels, tracks, and/or treads in one embodiment. In other embodiments, the ground vehicle 2010 may be a legged robot. In some embodiments, the ground vehicle 2010 may be used as a base station for one or more UAVs 2002. In some embodiments, one or more aerial devices, such as the UAV 2002, a balloon, or the like, may be tethered to the ground vehicle 2010 or a deployable tether controlled by a sensor operator 2008. In some embodiments, one or more trace gas sensors may be located in one or more stationary monitoring devices 2026. The one or more stationary monitoring devices may be located proximate one or more potential gas sources 2020, 2022. In some embodiments, the one or more stationary monitoring devices may be relocatable.

In some embodiments, the one or more vehicles 2002, 2004, 2006, 2010 and/or stationary monitoring devices 2026 may transmit data including trace gas data to a Ground Control Station (GCS) 2012. The GCS may include a display 2014 for displaying the trace gas concentrations to a GCS user 2016. The GCS user 2016 may be able to take corrective action if a gas leak 2024 is detected, such as by ordering a repair of the source 2020 of the trace gas leak. The GCS user 2016 may be able to control or dictate the movement of the one or more vehicles 2002, 2004, 2006, 2010 in order to confirm a presence of a trace gas leak in some embodiments.

In some embodiments, the GCS 2012 may transmit data to a cloud server 2018. In some embodiments, the cloud server 2018 may perform additional processing on the data. In some embodiments, the cloud server 2018 may provide third party data to the GCS 2012, such as wind speed, temperature, pressure, weather data, or the like.

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention herein disclosed by way of examples should not be limited by the particular disclosed embodiments described above.

What is claimed is:

1. An emissions characterization system (800) comprising:
one or more trace gas emission monitoring devices (802, 804, 806, 808, 810, 812, 814, 816, 818, 820);
wherein the one or more trace gas emission detection devices are configured to be connected via a communication network (822); and
wherein the one or more emission detection devices are further configured to perform one or more of: alert, verify, and quantify anomalous trace gas emissions.

2. The system (800) of claim 1, wherein the communication network (822) comprises a mesh network.

3. The system (800) of claim 2, wherein the mesh network comprises a wired and wireless network.

4. The system (800) of claim 1, wherein the one or more trace gas emission monitoring devices comprise one or more satellite monitoring systems (802, 804), wherein the one or more satellite monitoring systems (802, 804) are configured to generate data from one or more satellites.

5. The system (800) of claim 1, wherein the one or more trace gas emission monitoring devices comprise one or more aerial vehicle measurement systems (806, 808), wherein the one or more aerial vehicle measurement systems (806, 808) comprise one or more trace gas sensors configured to generate data on trace gas concentrations.

6. The system (800) of claim 5, wherein the one or more aerial vehicle measurement systems (806, 808) comprise a manned aerial vehicle (2004).

7. The system (800) of claim 5, wherein the one or more aerial vehicle measurement systems (806, 808) comprise an unmanned aerial vehicle (UAV) (2002).

8. The system (800) of claim 1, wherein the one or more trace gas emission monitoring devices comprise one or more fixed monitor measurement systems (810, 812), wherein the one or more fixed monitor measurement systems (810, 812) comprise one or more trace gas sensors configured to generate data on trace gas concentrations, and wherein the one or more trace gas sensors are fixed in a location.

9. The system (800) of claim 1, wherein the one or more trace gas emission monitoring devices comprise one or more robotics measurement systems (814, 816), wherein the one or more robotics measurement systems (814, 816) comprise one or more trace gas sensors configured to generate data on trace gas concentrations.

10. The system (800) of claim 9, wherein the one or more robotics measurement systems (814, 816) comprise one or more ground robotics.

11. The system (800) of claim 9, wherein the one or more robotics measurement systems (814, 816) comprise one or more surface robotics.

12. The system (800) of claim 1, wherein the one or more trace gas emission monitoring devices comprise one or more autonomous gas measurement systems (818, 820), wherein the one or more autonomous gas measurement systems (818, 820) comprise one or more trace gas sensors configured to generate data on trace gas concentrations.

13. The system (800) of claim 1, further comprising:
an emissions characterization module (826) configured to receive data (824) from the communication network (822) comprising the one or more trace gas emission monitoring devices (802, 804, 806, 808, 810, 812, 814, 816, 818, 820).

14. The system (800) of claim 13, wherein the emissions characterization module (826) is configured to alert, verify, and quantify anomalous trace gas emissions activities.

15. The system (800) of claim 13, wherein the emissions characterization module (826) is configured to determine if the anomalous trace gas emissions activities are at least one of: a false positive (607) and a result of a trace gas leak.

16. The system (800) of claim 15, wherein the emissions characterization module (826) is configured to provide a feedback loop to the communication network (822) comprising the one or more trace gas emission monitoring devices (802, 804, 806, 808, 810, 812, 814, 816, 818, 820) based on the determination to increase an accuracy of the communication network (822) and reduce a number of false positives (607).

17. The system (800) of claim 15, wherein the emissions characterization module (826) is configured to at least one of: shut off a gas flow (420) and initiate a repair protocol (418) if the determined anomalous trace gas emissions activities are the result of a trace gas leak.

18. The system (800) of claim 15, wherein the emissions characterization module (826) is configured to activate an emissions alarm if the determined anomalous trace gas emissions activities are the result of a trace gas leak.

19. A method (600, 610) comprising:
detecting emissions of one or more trace gasses (606) by at least one of: one or more satellite emissions surveys (602), one or more distributed sensors in a mesh continually monitoring for leaks (604), and one or more manned aircraft emission surveys (612);
triggering a high-resolution sensor deployment in response to detected emissions of the one or more trace gasses (608);
categorizing the detected emissions of the one or more trace gasses as a false positive (612) if the triggered high-resolution sensor deployment does not detect any further emissions;
performing localization and quantification of the emissions (614) if the triggered high-resolution sensor deployment detects further emissions; and
reconfiguring at least one of: a mesh network sensitivity and a mesh network density (616) if the triggered high-resolution sensor deployment detects further emissions.

20. The method (600, 610) of claim 19, wherein the triggered high-resolution sensor deployment further comprises a high-resolution survey from an unmanned aerial vehicle (UAV) comprising one or more trace gas sensors.

21. A method comprising:
receiving emissions data from at least one of: one or more satellite emissions surveys (602), a distributed sensor mesh (603), and one or more manned aircraft emission surveys (612); and
generating a database comprising at least one of: geographical regions that have higher than average trace gas emissions, equipment groups that have higher than average trace gas emissions, equipment components that have higher than average trace gas emissions, and equipment manufacturers that have higher than average trace gas emissions.

22. The method of claim 21, further comprising:
determining predictive maintenance from the generated database to reduce future trace gas emissions.

23. The method of claim 22, further comprising:
determining at least one of: equipment groups and areas that will be more likely to emit trace gasses based on the generated database.
